(19) United States
(12) Patent Application Publication
(10) Pub. No.: US 2009/0274764 A1
(43) Pub. Date: Nov. 5, 2009

(54) Hollow Foam Beads for Treatment of Glioblastoma

(76) Inventors: Hiep Q. Do, Sinking Spring, PA (US); Mark Timmer, Jersey City, NJ (US); Murty N. Vyakarnam, Springfield, NJ (US); Joel Rosenblatt, Royersford, PA (US); Han Cui, Basking Ridge, NJ (US); Scott Wadsworth, New Hope, PA (US); Janel E. Young, Baltimore, MD (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/426,405

(22) Filed: Apr. 20, 2009

Related U.S. Application Data
(60) Provisional application No. 61/049,027, filed on Apr. 30, 2008.

(51) Int. Cl. A61K 9/14

(52) U.S. Cl. 424/489

(57) ABSTRACT

Compositions and methods for the treatment of tumors are disclosed. Specifically, the present invention provides hollow foam beads having a chemotherapeutic agent incorporated therein. A method of making such beads is disclosed. In addition, a method for treating a glioblastoma tumor and other types of tumors with the compressible hollow foam beads is disclosed.

### CROSS REFERENCE TO RELATED APPLICATIONS

This application claims priority from U.S. Provisional Application Ser. No. 61/049,027 filed on Apr. 30, 2008.

### FIELD OF THE INVENTION

The invention relates to compositions and methods for the treatment of brain cancer. More specifically, compositions and methods for the treatment of glioblastoma.

### BACKGROUND OF THE INVENTION

The standard therapy for brain tumors is surgical resection (if possible) followed by radiation and chemotherapy. Delivery of chemotherapeutic agents to the resection site is limited by toxic side effects or the inability of many compounds to transit the blood brain barrier at effective concentrations. For glioblastoma multiforme, the most lethal brain cancer, tumor regrowth generally occurs in close proximity to where the initial tumor was resected. One characteristic of glioblastoma is that the tumor infiltrates surrounding brain tissue and thus is difficult to completely excise during surgery.

Local delivery of chemotherapeutic agents from the resection cavity provides a method for delivering high concentrations of chemotherapeutic agents to where they are needed while avoiding systemic side effects. This approach was successfully demonstrated with the Gliadel wafer (marketed by Guilford Pharmaceuticals-MGI Pharma), which locally delivers the DNA alkylating agent BCNU from a polyanhydride wafer. Sustained BCNU delivery is reported to last for 2-3 weeks. Clinical studies have shown that the use of Gliadel extends the mean survival of glioblastoma patients from 11.6 to 13.9 months. Due to the brittleness and difficulty in processing polyanhydrides, these wafers are difficult to handle. They are rigid and have dimensions of 1.5 cm diameter×1 mm thick. The resection cavity is lined with up to 8 wafers. Gliadel has demonstrated that local delivery from a biodegradable polymer brings therapeutic benefit in treating glioblastoma.

There still exists a need for more effective drugs or drug combinations and delivery vehicles with improved handling properties.

### SUMMARY OF THE INVENTION

Accordingly, novel compressible hollow foam beads are disclosed. The foam beads comprise a biocompatible, bioabsorbable elastomeric copolymer and at least one chemotherapeutic agent.

Another aspect of the present invention is a method of manufacturing the above-described beads. In that method, a solution of a biocompatible, bioabsorbable elastomeric copolymer in a solvent is prepared. A chemotherapeutic agent is added to the solution. Drops of the solution are added to a liquid nitrogen bath to provide hollow frozen drops. The drops are lyophilized to provide compressible hollow foam beads.

Yet another aspect of the present invention is a method of treating a glioblastoma or other cancer using the foam beads of the present invention.

The and other aspects and advantages of the present invention will become more apparent from the following description and accompanying drawings

### BRIEF DESCRIPTION OF THE DRAWINGS

FIGS. 1A-C are scanning electron microscope images of the beads of the present invention illustrating respectively a bead, a cross-section showing an inner cavity, and a section of the surface.

### DETAILED DESCRIPTION OF THE INVENTION

The present invention provides compressible hollow foam beads having a chemotherapeutic agent incorporated therein, a method of manufacturing such beads, and a method for treating a glioblastoma tumor with one or more of said compressible hollow foam beads.

The soft, elastomeric, compressible hollow foam beads of the present invention are useful for placement in a glioblastoma surgical resection cavity. The foam beads are easily compressed to conform to the irregular shape of a resection cavity, thereby maximizing contact surface area with surrounding tissue where tumor cells may not have been completely excised. The foam is highly porous (over 90% void volume), such that it can be freely compressed from its rest state to as little as 1/10^{th} its original volume or less. The foam bead is administered by first mechanically compressing it and then releasing it within the resection cavity. The foam beads can be compressed between the surgeons fingers or by using a surgical instrument, including for example, forceps. Although less desirable, the foam beads can be inserted by packing into the cavity without first compressing them. Once released, the beads expand to fill and conform to the shape of the cavity. The foam of the foam beads is soft so that it exerts a minimal mechanical compression against the edges of the resection cavity. The foam makes intimate contact with all or substantially all of the surfaces of the resection cavity such that preloaded chemotherapeutic agent in the foam beads can diffuse to all foam contact surfaces.

The foam beads are prepared by first preparing a solution or suspension of a biocompatible, biodegradable elastomeric polyester copolymer, a chemotherapeutic agent, and a suitable solvent.

Suitable biocompatible biodegradable elastomeric copolymers include, but are not limited to copolymers of epsilon-caprolactone and glycolide (preferably having a mole ratio of epsilon-caprolactone to glycolide of from about 30:70 to about 70:30, preferably 35:65 to about 65:35, and more preferably 45:55 to 35:65); elastomeric copolymers of epsilon-caprolactone and lactide, including L-lactide, D-lactide blends thereof or lactic acid copolymers (preferably having a mole ratio of epsilon-caprolactone to lactide of from about 35:65 to about 65:35 and more preferably 45:55 to 30:70;) elastomeric copolymers of p-dioxanone (1,4-dioxan-2-one) and lactide including L-lactide, D-lactide and lactic acid (preferably having a mole ratio of p-dioxanone to lactide of from about 40:60 to about 60:40); elastomeric copolymers of epsilon-caprolactone and p-dioxanone (preferably having a mole ratio of epsilon -caprolactone to p-dioxanone of from about 30:70 to about 70:30); elastomeric copolymers of p-dioxanone and trimethylene carbonate (preferably having a mole ratio of p-dioxanone to trimethylene carbonate of from about 30:70 to about 70:30); elastomeric copolymers of trimethylene carbonate and glycolide (preferably having a mole ratio of trimethylene carbonate to glycolide of from about 30:70 to about 70:30); elastomeric copolymer of trimethylene carbonate and lactide including L-lactide, D-lactide, blends thereof or lactic acid copolymers (preferably having a mole ratio of trimethylene carbonate to lactide of from about 30:70 to about 70:30) and blends thereof. In one embodiment, the biocompatible, biodegradable elastomeric copolymers are copolymers of epsilon-caprolactone and glycolide. In another embodiment, the biocompatible, biodegradable elastomeric copolymers are copolymers of epsilon-caprolactone and glycolide having a mole ratio of epsilon-caprolactone to glycolide of from about 45:55 to 35:65. In yet another embodiment, the biocompatible, biodegradable elastomeric copolymers is a copolymer of epsilon-caprolactone and glycolide having a mole ratio of epsilon-caprolactone to glycolide of about 35:65.

The biodegradable copolymers readily break down into small segments when exposed to moist body tissue. The segments then either are absorbed by the body, or passed by the body. More particularly, the biodegraded segments do not elicit permanent chronic foreign body reaction, because they are absorbed by the body or passed from the body, such that no permanent trace or residual of the segment is retained by the body.

In addition to the polymers and copolymers as described above, various chemotherapeutic agents may be incorporated into the solution to prepare the chemotherapeutic agent-loaded foam beads. Chemotherapeutic agents include, but are not limited to radiation sensitizers, such as Zarnestra or temozolomide; cytotoxic agents, such as paclitaxel; agents that interfere with DNA replication, such as DNA alkylating agents; cytostatic agents such as rapamycin; angiogensis inhibitors, inhibitors of immune tolerizing cytokines, and chemotaxis inhibitors such as TGF-beta receptor kinase inhibitors, and other conventional chemotherapeutic agents, and combinations thereof. Of particular interest are combinations of drugs that can inhibit the tumor through multiple pathways. In one embodiment, the chemotherapeutic agent is rapamycin.

Suitable solvents for preparing the solutions include but are not limited to formic acid, ethyl formate, acetic acid, hexafluoroisopropanol (HFIP), cyclic ethers (i.e. THF, DMF, and PDO), acetone, acetates of C2 to C5 alcohol (such as ethyl acetate and t-butylacetate), glyme (i.e. monoglyme, ethyl glyme, diglyme, ethyl diglyme, triglyme, butyl diglyme and tetraglyme) methylethyl ketone, dipropyleneglycol methyl ether, lactones (such as γ-valerolactone, δ-valerolactone, β-butyrolactone, γ-butyrolactone) 1,4-dioxane, 1,3-dioxolane, 1,3-dioxolane-2-one (ethylene carbonate), dimethlycarbonate, benzene, toluene, benzyl alcohol, p-xylene, naphthalene, tetrahydrofuran, N-methyl pyrrolidone, dimethylformamide, chloroform, 1,2-dichloromethane, morpholine, dimethylsulfoxide, hexafluoroacetone sesquihydrate (HFAS), anisole and mixtures thereof. In one embodiment the solvent is 1,4-dioxane.

Chemotherapeutic agent-loaded foam beads are prepared by either co-dissolving or suspending the chemotherapeutic agent in a polymer solution, adding drops of the solution or suspension to a liquid nitrogen bath thereby freezing the droplets, and lyophilizing the droplets to yield the compressible hollow foam beads. The resulting foam beads contain the chemotherapeutic agent encapsulated or contained in the foam. This enables sustained release of the drug.

A solution or suspension of the polymer and chemotherapeutic agent in the solvent is prepared using standard, conventional techniques. As a general guideline (although not limited thereto) the amount of polymer in the solution can vary from about 1% to about 20% by weight. In one embodiment, the amount of polymer in the solution is about 1% to about 10% by weight of the solution. Typically, the amount of chemotherapeutic agent in the solution is from about 0.001 percent to about 30 percent by weight of the solution. In one embodiment, the amount of chemotherapeutic agent in the solution is about 0.3 percent to about 30 percent by weight of the solution. As mentioned above, the chemotherapeutic agent may be dissolved or in suspension. The amount of the chemotherapeutic agent will be sufficient to effectively provide a therapeutically effective amount of the agent when the beads are implanted in a patient.

After the solution or suspension is prepared as described above, the solution or suspension is then added dropwise into a bath containing liquid nitrogen using an injection system. Although not preferred, it may be possible to use other cryogenic fluids including but not limited to liquid gases or mixtures of liquid gases, such as liquid helium. The size of the bead is controlled by the pressure (for example, hydraulic pressure) and nozzle diameter. Typically, these beads are substantially spherical in shape and have a sufficiently effective size, for example greater than 100 microns in diameter and less than 10 mm in diameter or more typically in the 100 microns to 3 mm diameter range, although other sizes and combinations of sizes may be used. The frozen bead has a visible hollow pore after removal from liquid nitrogen and prior to the freeze-drying step. The frozen hollow beads are removed from the liquid nitrogen and then placed in a freeze-dryer chamber that is pre-cooled to −17° C. The beads are subsequently lyophilized in a conventional process to remove the solvent from the frozen beads by phase separation. The beads are optionally sorted and singulated by size.

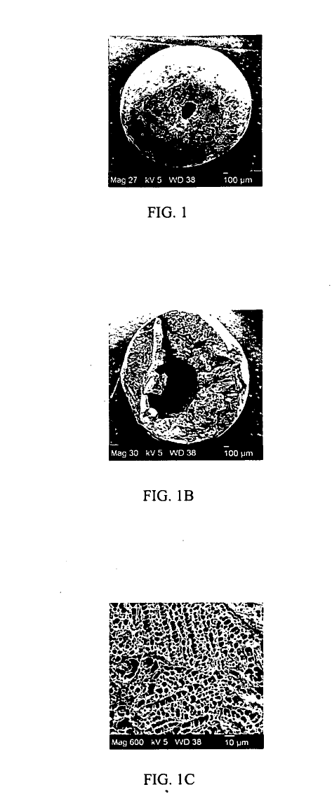

Referring to FIGS. 1A-C, Scanning electron microscope (SEM) analysis was performed on 35/65 PCL/PGA porous bead samples loaded with 28% rapamycin. The cross-sectioned samples were prepared by freezing the samples in a bath of liquid nitrogen and cross-sectioning the beads with a sharp blade. The SEM samples were mounted on a microscope stud and coated with a thin layer of gold using an EMS 550 sputter coater. The beads were analyzed using a JEOL JSM-5900LV SEM. FIG. 1A shows bead surface magnified 27×; FIG. 1B, bead cross-section magnified 30×; and, FIG. 1C, bead surface magnified 600×.

The observed morphology for the bead samples showed a spherical shape with a smooth textured surface. The diameter of the beads was approximately 3.0 mm. The SEM analysis indicated a bead with a large single pore, which was approximately 400 microns in diameter (FIG. 1A). The SEM images of the cross-sections showed a large circular cavity (approx. 1 mm in diameter) located in the center of the beads (FIG. 1B). Analysis of the bead surfaces showed some localized areas with small porous openings approximately≦5 microns in diameter (FIG. 1C).

The foam beads may be packaged and/or stored in a conventional dry nitrogen environment at room temperature and protected from light. When packaged, the packages will preferably be made from conventional gas-tight materials, such as a metal foil/polymr laminate with hermetic seals. The foam beads are preferably sterile, and may be sterilized using conventional sterilization processes suitable for such materials or may be manufactured and packaged aseptically using conventional techniques.

A therapeutically effective amount of the foam beads of the present invention is used in a surgical procedure and this will be determined by the surgeon based upon various patient characteristics and medical parameters including the typed and size of the tumor, etc. The size of the beads of the present invention that are used in the surgical procedures of the present invention will be that of a range of sufficiently effective single sizes or may consist of a population of foam beads having a sufficiently effective size distribution in a range.

The chemotherapeutic agent-loaded hollow compressible foam beads of the present invention are useful in the treatment of glioblastoma. One method of the present invention for treating a glioblastoma tumor using the foam beads of the present invention is described as follows. Initially, a glioblasoma tumor is resected in a patient. Then one or more compressible hollow foam beads of the present invention consisting of a biocompatible, bioabsorbable elastomeric polyester polymer (copolymer) and a chemotherapeutic agent are provided. The surgeon then administers to the resection site a therapeutically effective amount of the compressible hollow foam bead(s) to sufficiently fill the cavity at the resection site. The compressible hollow foam bead(s) are administered to the glioblastoma resection site by first compressing the bead(s), then placing the bead(s) in the site, and allowing the bead(s) to resume to their original spherical shape or a shape conforming to the surface of the resection site or possibly to remain in a compressed state, or a combination thereof. Although it is preferred to fill in the entire cavity, the surgeon may in the surgeon's discretion fill in a part of the cavity. The surgical site is then closed in a conventional manner. The method of treating glioblastoma as described above may also be used in a treatment protocol in addition to or in combination with one or more of the current conventional standard of care treatments for glioblastoma, including treatment with radiation, such as x-ray and chemotherapy.

The foam beads of the present invention may be used to treat other tumors and types of cancer as well, including cancers of the breast, prostate, ovary, colon, head and neck, and neuroendocrine organs. The surgical procedures are similar to the procedure described herein wherein the beads are loaded into a surgical resection site or the procedures may be adapted to the location and type of cancerous tumor. The chemotherapeutic agents selected would be conventional agents and other agents developed to treat the particular types of cancer. It is also possible also to load the beads of the present invention adjacent to a tumor site without surgically removing the tumor.

The use of small compressible foam drug-loaded beads of the present invention provides advantages over presently known and used local intracranial drug-loaded therapy (Gliadel®) supplied as 1.45 cm diameter×1 mm thick biodegradable polyanhydride wafers. The number of such wafers, and hence the therapeutic dose, that can be implanted is limited by the size and geometry of the tumor resection site. Also, the degree of contact between the brain tissue and the wafers is limited by the size and geometry of the wafers. In contrast, the use of much smaller, e.g., ˜3 mm, compressible foam beads of the present invention allows the surgeon to implant as many beads as required to more completely fill the tumor resection site. Also of importance to this invention is that the foam can be easily compressed to conform to the irregular shape of a resection cavity as well as maximize contact surface area with surrounding tissue where tumor cells may not have been excised. The foam is soft, so that it exerts a minimal mechanical compression against the edges of the resection cavity, in contrast to more rigid polyanhydride wafers. The foam makes intimate contact with all or substantially all of the surfaces of the resection cavity such that preloaded drug can diffuse to all foam contact surfaces, again in contrast to larger, more rigid polyanhydride wafers of fixed geometry.

The following examples are illustrative of the principles and practice of this invention, although not limited thereto. Numerous additional embodiments within the scope and spirit of the invention will become apparent to those skilled in the art once having the benefit of this disclosure.

### EXAMPLE 1

### Rapamycin-Loaded Polymer Beads.

To prepare the rapamycin-loaded beads, a solution containing 5% by weight of a 35% polycaprolactone/65% polyglycolic acid polymer solution in 1,4 Dioxane solvent was prepared. This polymer solution was heated to 60 degrees C. for 4 hrs with continuous stirring to ensure complete dissolution of the polymer. The solution was then filtered through an extra coarse Pyrex fritted filter prior to use.

Preliminary experiments determined that the maximum drug loading capacity of the beads was about 28%, by weight, therefore three concentrations of rapamycin in the polymer solution were prepared, at a target of 0.3, 3, and, 28% by weight. Rapamycin was incorporated into the polymer solution (0.3, 3, 28% by weight) at room temperature with continuous stirring. The drug dissolved in polymer solution instantaneously. The drug-loaded solution was then added dropwise through a disposable glass pipette into a dewar flask containing liquid nitrogen to form frozen beads. These frozen beads were placed in an aluminum tray and lyophilized to remove the solvent. The frozen hollow beads are removed from the liquid nitrogen and then placed in a freeze-dryer chamber that is pre-cooled to −17° C. The beads are subsequently lyophilized to remove the solvent from the frozen beads by phase separation. Beads were stored at room temperature under nitrogen gas and protected from light until use. The actual measured rapamycin concentrations were 0.23%, 2.3%, and 28%.

### EXAMPLE 2

### In Vitro Activity of Rapamycin.

The 9L gliosarcoma cell line was obtained from Dr. M. Barker at the University of California at San Francisco Brain Tumor Research Center (San Francisco, Calif., USA). The cells were maintained in tissue culture in Dulbecco's minimum essential medium with 10% fetal bovine serum, streptomycin (80.5 units/ml), penicillin (base; 80.5 units/ml), and 1% L-glutamine (all products from GIBCO laboratories, Grand Island, N.Y., USA). Cells were maintained in a humidified atmosphere of 5% CO₂ at 37° C. The cells were grown to confluence, detached with 0.25% trypsin in Dulbecco's phosphate-buffered saline, and resuspended in medium.

Inhibition of tumor proliferation was tested against the rodent 9L glioma. Cells were plated at 10,000 cells/well in 24-well plates with increasing concentrations of rapamycin, ranging from 0.01 microgram/ml to 10 microgram/ml. The cells were counted after 5-days, using a cell counter and compared with control cells receiving no rapamycin. The data were analyzed using the two-tailed Student's t-test.

### Results

Rapamycin was cytotoxic to 9L cells, causing a 34% growth inhibition at 0.01 microgram/ml and 62% growth inhibition at 10 microgram/ml.

### EXAMPLE 3

### In Vivo Efficacy Testing of Rapamycin Loaded Beads in a Rodent Model.

### Cells

The 9L gliosarcoma cell line was obtained from Dr. M. Barker at the University of California at San Francisco Brain Tumor Research Center (San Francisco, Calif., USA). The cells were maintained in tissue culture in Dulbecco's minimum essential medium with 10% fetal bovine serum, streptomycin (80.5 units/ml), penicillin (base; 80.5 units/ml), and 1% L-glutamine (all products from GIBCO laboratories, Grand Island, N.Y., USA). Cells were maintained in a humidified atmosphere of 5% CO₂ at 37° C. The cells were grown to confluence, detached with 0.25% trypsin in Dulbecco's phosphate-buffered saline, and resuspended in medium.

### Animals

Female Fisher 344 rats weighing 180 to 220 g were purchased from Charles River Laboratories (Wilmington, Mass., USA). The animals were kept in standard animal facilities with 3 or 4 rats per cage, and given free access to rat chow and water. They were housed in accordance with the policies and principles of laboratory care of the institutional Animal Care and Use Committee. Five animals per group were used for the toxicity studies.

### Intracranial Tumor Implantation

Rats were anesthetized with an intraperitoneal injection of 2 to 4 ml/kg of a stock solution containing ketamine hydrochloride (25 mg/ml), xylazine (2.5 mg/ml), and ethanol in a sterile 0.9% NaCl solution. The heads were shaved and disinfected with a 70% ethanol and povidone-iodine solution. After a midline scalp incision, the galea overlying the left cranium was swept laterally. With the aid of an operating microscope, a 3-mm burr hole was made over the left parietal bone, with its center 2 to 3 mm posterior to the coronal suture and 3 to 4 mm lateral to the sagittal suture. Great care was taken to avoid injury to the dura mater. The rats were then placed in a stereotactic frame, and 1×10² 9L glioma cells were implanted, with or without a rapamycin-loaded polymer bead prepared as described in Example 1. After ensuring hemostasis, the wound was closed with surgical staples.

### Bead Implantation

In animals not receiving tumor cells, following burr hole placement, the dura mater and underlying brain parenchyma were opened using a No. 11 surgical blade. Then, with the aid of an operating microscope, one 3 mg bead (approx. 3 mm diameter) was placed into the brain parenchyma at a depth of approximately 1 mm below the dura. After ensuring hemostasis, the skin was closed with surgical staples. In tumor-bearing animals, beads were either implanted at the time of tumor implantation (day 0) or surgical wounds were reopened and beads were implanted five days after tumor implantation. Toxicity was assessed for 40 days.

### In Vivo Rapamycin Bead Toxicity.

To determine the maximally tolerated rapamycin loading dose, 20 rats, evenly divided into 4 groups, underwent intracerebral implantation of beads containing 28%, 2.3%, and 0.23% rapamycin (prepared in Example 1). Animals were closely monitored for signs of toxicity, including wound healing problems, weight loss, failure to thrive, and neurological deficits.

### Results

When delivered intracranially (IC) to healthy rats, 0.23, 2.3, or 27.8% rapamycin-loaded beads (one 3 mg bead per rat) had no effect on weight gain, survival or gross histopathology of the brain. Therefore the 27.8% rapamycin-loaded beads were used for the initial efficacy studies (Example 4).

### EXAMPLE 4

In Vivo Efficacy Testing of Rapamycin Loaded Beads in a Rodent Model.

### Cells

The 9L gliosarcoma cell line was obtained from Dr. M. Barker at the University of California at San Francisco Brain Tumor Research Center (San Francisco, Calif., USA). The cells were maintained in tissue culture in Dulbecco's minimum essential medium with 10% fetal bovine serum, streptomycin (80.5 units/ml), penicillin (base; 80.5 units/ml), and 1% L-glutamine (all products from GIBCO laboratories, Grand Island, N.Y., USA). Cells were maintained in a humidified atmosphere of 5% CO₂ at 37° C. The cells were grown to confluence, detached with 0.25% trypsin in Dulbecco's phosphate-buffered saline, and resuspended in medium.

### Animals

Female Fisher 344 rats weighing 180 to 220 g were purchased from Charles River Laboratories (Wilmington, Mass., USA). The animals were kept in standard animal facilities with 3 or 4 rats per cage, and given free access to rat chow and water. They were housed in accordance with the policies and principles of laboratory care of the institutional Animal Care and Use Committee. Eight animals per group were used for the efficacy studies.

### Intracranial Tumor Implantation

Rats were anesthetized with an intraperitoneal injection of 2 to 4 ml/kg of a stock solution containing ketamine hydrochloride (25 mg/ml), xylazine (2.5 mg/ml), and ethanol in a sterile 0.9% NaCl solution. The heads were shaved and disinfected with a 70% ethanol and povidone-iodine solution. After a midline scalp incision, the galea overlying the left cranium was swept laterally. With the aid of an operating microscope, a 3-mm burr hole was made over the left parietal bone, with its center 2 to 3 mm posterior to the coronal suture and 3 to 4 mm lateral to the sagittal suture. Great care was taken to avoid injury to the dura mater. The rats were then placed in a stereotactic frame, and 1×10² 9L glioma cells were implanted, with or without a rapamycin-loaded polymer bead prepared as described in Example 1. After ensuring hemostasis, the wound was closed with surgical staples.

### Bead Implantation

In animals not receiving tumor cells, following burr hole placement, the dura mater and underlying brain parenchyma were opened using a No. 11 surgical blade. Then, with the aid of an operating microscope, one 3 mg bead (approx. 3 mm diameter) was placed into the brain parenchyma at a depth of approximately 1 mm below the dura. After ensuring hemostasis, the skin was closed with surgical staples. In tumor-bearing animals, beads were either implanted at the time of tumor implantation (day 0) or surgical wounds were reopened and beads were implanted five days after tumor implantation. Survival was then assessed. Animals surviving until day 100 were considered to be cured.

### In Vivo Rapamycin Bead Efficacy.

To determine the efficacy of intracranially implanted rapamycin-loaded beads in the rat intracranial 9L glioblastoma model, 40 rats, evenly divided into 5 groups, underwent intracerebral implantation of beads containing approximately 28% rapamycin (prepared as in Example 1) either at the time of tumor implantation (day 0) or day 5 after tumor implantation, placebo beads without rapamycin on day 0, or no beads (control). The data are compared to the results obtained by implantation of 10 mm diameter×1 mm thick poly(L-lactic acid) poly(lactic co-glycolic) polymer discs containing 3.8% 1,3-bis (2-chloroethyl)-1-nitrosurea (BCNU, the active ingredient in the Gliadel® wafer), prepared according to Kim et al., J. Contr. Rel., 2007, 123:172-78. Animals received one bead, one disc, or no treatment. For efficacy, animals survival was evaluated for 100 days. Survival data were analyzed with the log-rank (Mantel-Cox) test in a Kaplan-Meier nonparametric analysis performed using statistical software.

### Results

Results are displayed in the Table 1 and Graph 1 below.

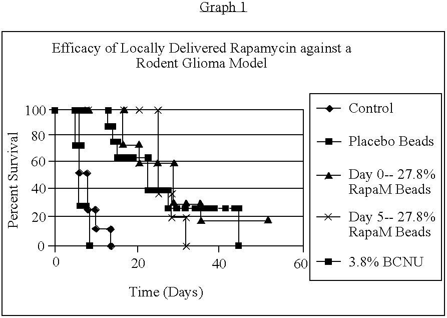

TABLE 1
Statistical Significance

| | | | | | 3.8% |
|---|---|---|---|---|---|
| | Control | Placebo | Day 0 Rapa | Day 5 Rapa | BCNU |
| Control | xxx | 0.1117 | <0.0001 | <0.0001 | <0.0001 |
| Placebo | 0.1117 | xxx | 0.0002 | 0.0002 | 0.0001 |
| Day 0 Rapa | <0.0001 | 0.0002 | xxx | 0.741 | 0.6085 |
| Day 5 Rapa | <0.0001 | 0.0002 | 0.741 | xxx | 0.7972 |
| 3.8% BCNU | <0.0001 | <0.0001 | 0.6085 | 0.7972 | xxx |

Rapamycin, delivered intracranially at the time of tumor implantation in the rat 9L glioma model, was as effective as intracranially delivered 1,3-bis (2-chloroethyl)-1-nitrosurea (BCNU, the active ingredient in the Gliadel® wafer) at prolonging survival in the rat 9L glioma model. All treatments were statistically significantly different from untreated rats or rats receiving placebo beads (beads without rapamycin). There were no statistical differences between the treatments.

### EXAMPLE 5

In Vivo Efficacy Testing of Rapamycin Loaded Beads in a Rodent Model With and Without X-ray Therapy.

In the following example, intracranial rapamycin beads were delivered as described in Example 4. On day 0 or day 5 animals were additionally treated with and without X-ray therapy (XRT, 20 Gy, delivered on day 5). Results are presented in Graph 2 and Table 2 below.

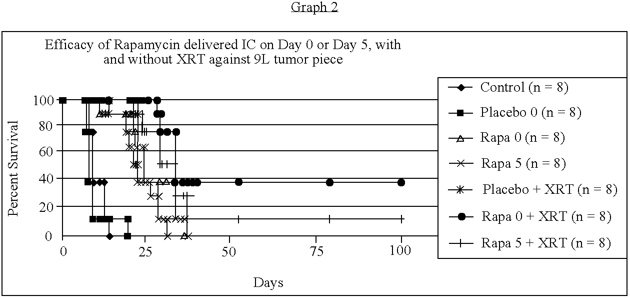

TABLE 2
Statistical Significance

| | Control- | | | | | Rapa 0 + | Rapa 5 + |
|---|---|---|---|---|---|---|---|
| | No RX | Placebo 0 | Rapa 0 | Rapa 5 | XRT | XRT | XRT |
| Control-No RX | xxx | 0.4393 | 0.0004 | 0.0001 | <0.0001 | <0.0001 | 0.0001 |
| Placebo day 0 | 0.4393 | xxx | 0.0001 | 0.0003 | <0.0001 | 0.0008 | <0.0001 |
| Rapamycin day 0 | 0.0004 | 0.0001 | xxx | 0.1799 | 0.6895 | 0.0484 | 0.1342 |
| Rapamycin day 5 | 0.0001 | 0.0003 | 0.1799 | xxx | 0.2997 | 0.01 | 0.01 |
| XRT | <0.0001 | <0.0001 | 0.6895 | 0.2997 | xxx | 0.0045 | 0.1407 |
| Rapa day 0 + XRT | <0.0001 | 0.0008 | 0.0484 | 0.01 | 0.0045 | xxx | 0.1868 |
| Rapa day 5 + XRT | <0.0001 | 0.0001 | 0.1342 | 0.01 | 0.1407 | 0.1868 | Xxx |

This example shows that IC rapamycin was as effective as XRT that was given in a dose to mimic that received by human patients. The data further shows that the combination of IC rapamycin on day 0+XRT on day 5 was statistically superior to either treatment alone and resulted in the survival of ⅜ rats (37.5%) to 100 days, which is considered cured. The combination of IC rapamycin on day 5+XRT on day 5 was statistically superior to IC rapamycin alone on day 5 and resulted in the survival of ⅛ rats (12.5%) to 100 days. There was a highly significant difference for all treatments compared to control or placebo treated animals.

### EXAMPLE 6

### In Vivo Dose Response of Rapamycin Loaded Beads in a Rodent Model.

Animals were treated with 0, 0.2, 2.2, or 28% rapamycin-loaded beads, prepared as described in Example 1, using methods described in Example 4. Beads were delivered on day 0 concurrent with tumor implantation or on day 5 post tumor implantation, and were compared to the effect of daily systemic IP doses of rapamycin. 88 rats were intracranially implanted on Day 0 with 9L gliosarcoma. Animals were then randomly divided into 11 groups and received one of the following treatments:
Group 1—No Treatment, Tumor Only (n=8)
Group 2—Day 0—Local implantation of Placebo Beads (n=8)
Group 3—Day 0—Local implantation of 28% Rapamycin Beads (n=8)
Group 4—Day 0—Local implantation of 2.2% Rapamycin Beads (n=8)
Group 5—Day 0—Local implantation of 0.2% Rapamycin Beads (n=8)
Group 6—Day 0—Systemic Dose of Rapamycin (dose TBD) (n=8)
Group 7—Day 5—Local implantation of Placebo Beads (n=8)
Group 8—Day 5—Local implantation of 28% Rapamycin Beads (n=8)
Group 9—Day 5—Local implantation of 2.2% Rapamycin Beads (n=8)
Group 10—Day 5—Local implantation of 0.2% Rapamycin Beads (n=8)
Group 11—Day 5—Systemic Dose of Rapamycin (2 mg/kg given daily by intraperitoneal (IP) injection in DMSO for 30 days) (n=8)

### Results

As seen previously (see Graphs 1 & 2, Tables 1 & 2), there was no difference in survival between untreated animals and those receiving placebo beads (data not shown). Animals receiving concurrent tumor and treatment had a mean survival of 13 days (placebo beads), 24 days (0.2% rapamycin beads), 28 days (2.2% rapamycin beads), 32 days (28% rapamycin beads) and 28 days (systemic rapamycin treatment). See Table 3. All treatment groups experienced an increase in survival as compared to the control group (p=0.0001 for 0.2%, p=0.0001 for 2.2%, and p=0.0014 for 28%). The group that received 28% rapamycin beads had an increased survival compared to those receiving 2.2% or 0.2% rapamycin beads (p=0.0434, p=0.0069, respectively). See Table 5.

Animals that received treatment 5 days after tumor implantation had a mean survival of 14 days (placebo beads), 19.5 days (0.2% rapamycin beads), 23 days (2.2% rapamycin beads), 24 days (28% rapamycin beads) and 29 days (systemic treatment). See Table 4. All Day 5 treatment groups experienced an increase in survival compared to controls (p=0.0004 for 0.2%, p=0.000 1 for 2.2%, and p=0.0001 for 28%). Those receiving 28% or 2.2% rapamycin beads had an increased survival as compared to 0.2% (p=0.0275 and p=0.0257, respectively). The systemic rapamycin delivery group and the 28% rapamycin bead group had similar survival (p=0.222) with the systemic delivery group having an increased survival compared to 2.2% (p=0.003) or 0.2% (p=0.0001). All groups treated at the same time as tumor implantation did significantly better than those implanted with rapamycin beads 5 days after establishment of the tumors. See Table 5.

TABLE 3
Survival when treatment was initiated concurrently with tumor
implantation.

| | % Survival | | | | |
|---|---|---|---|---|---|
| | | | | | Group 5 |
| | Group 1 | Group 2 | Group 3 | Group 4 | Day 0 - |
| | Day 0 - | Day 0 - 28% | Day 0 - 2.2% | Day 0 - 0.2% | Systemic Dose |
| | Placebo | Rapamycin | Rapamycin | Rapamycin | of Rapamycin |
| Day | Beads (n = 8) | Beads (n = 8) | Beads (n = 8) | Beads (n = 8) | (n = 8) |
| 0 | 100 | 100 | 100 | 100 | 100 |
| 12 | 100 | 100 | 100 | 100 | 100 |
| 12 | 75 | 100 | 100 | 100 | 100 |
| 13 | 75 | 100 | 100 | 100 | 100 |
| 13 | 62.5 | 100 | 100 | 100 | 100 |
| 14 | 62.5 | 100 | 100 | 100 | 100 |
| 14 | 50 | 100 | 100 | 100 | 100 |
| 15 | 50 | 100 | 100 | 100 | 100 |
| 15 | 50 | 100 | 100 | 87.5 | 100 |
| 16 | 50 | 100 | 100 | 87.5 | 100 |
| 16 | 12.5 | 100 | 100 | 87.5 | 100 |
| 17 | 12.5 | 100 | 100 | 87.5 | 100 |
| 17 | 0 | 100 | 100 | 87.5 | 100 |
| 18 | 0 | 100 | 100 | 87.5 | 100 |
| 18 | 0 | 100 | 100 | 87.5 | 100 |
| 19 | 0 | 100 | 100 | 87.5 | 100 |
| 19 | 0 | 100 | 100 | 75 | 100 |
| 20 | 0 | 100 | 100 | 75 | 100 |
| 20 | 0 | 87.5 | 100 | 75 | 100 |
| 21 | 0 | 87.5 | 100 | 75 | 100 |
| 21 | 0 | 87.5 | 100 | 75 | 100 |
| 22 | 0 | 87.5 | 100 | 75 | 100 |
| 22 | 0 | 87.5 | 100 | 75 | 100 |
| 23 | 0 | 87.5 | 100 | 75 | 100 |
| 23 | 0 | 87.5 | 100 | 75 | 100 |
| 24 | 0 | 87.5 | 100 | 75 | 100 |
| 24 | 0 | 87.5 | 100 | 62.5 | 100 |
| 25 | 0 | 87.5 | 100 | 62.5 | 100 |
| 25 | 0 | 87.5 | 75 | 37.5 | 75 |
| 26 | 0 | 87.5 | 75 | 37.5 | 75 |
| 26 | 0 | 62.5 | 62.5 | 25 | 75 |
| 27 | 0 | 62.5 | 62.5 | 25 | 75 |
| 27 | 0 | 62.5 | 62.5 | 25 | 75 |
| 28 | 0 | 62.5 | 62.5 | 25 | 75 |
| 28 | 0 | 62.5 | 50 | 25 | 50 |
| 29 | | 62.5 | 50 | 25 | 50 |
| 29 | | 62.5 | 50 | 25 | 50 |
| 30 | | 62.5 | 50 | 25 | 50 |
| 30 | | 62.5 | 12.5 | 25 | 37.5 |
| 31 | | 62.5 | 12.5 | 25 | 37.5 |
| 31 | | 62.5 | 0 | 12.5 | 37.5 |
| 32 | | 62.5 | | 12.5 | 37.5 |
| 32 | | 62.5 | | 12.5 | 37.5 |
| 33 | | 62.5 | | 12.5 | 37.5 |
| 33 | | 25 | | 12.5 | 37.5 |
| 35 | | 25 | | 12.5 | 37.5 |
| 35 | | 12.5 | | 12.5 | 25 |
| 40 | | 12.5 | | 12.5 | 25 |
| 40 | | 12.5 | | 12.5 | 12.5 |
| 41 | | 12.5 | | 12.5 | 12.5 |
| 41 | | 12.5 | | 12.5 | 0 |
| 42 | | 12.5 | | 12.5 | |
| 42 | | 12.5 | | 0 | |
| 84 | | 12.5 | | | |
| Mean Survival | 13 | 32 | 28 | 24 | 28 |
| (days) | | | | | |

TABLE 4
Survival when treatment was initiated 5 days after tumor
implantation.

| | % Survival | | | | |
|---|---|---|---|---|---|
| | | | | | Group 10 |
| | | Group 7 | | Group 9 | Day 5 - |
| | Group 6 | Day 5 - | Group 8 | Day 5 - | Systemic |
| | Day 5 - | 28% | Day 5 - 2.2% | 0.2% | Dose of |
| | Placebo | Rapamycin | Rapamycin | Rapamycin | Rapamycin |
| Day | Beads (n = 8) | Beads (n = 8) | Beads (n = 8) | Beads (n = 8) | (n = 8 |
| 0 | 100 | 100 | 100 | 100 | 100 |
| 12 | 100 | 100 | 100 | 100 | 100 |
| 12 | 100 | 100 | 100 | 100 | 100 |
| 13 | 100 | 100 | 100 | 100 | 100 |
| 13 | 100 | 100 | 100 | 100 | 100 |
| 14 | 100 | 100 | 100 | 100 | 100 |
| 14 | 75 | 100 | 100 | 100 | 100 |
| 15 | 75 | 100 | 100 | 100 | 100 |
| 15 | 12.5 | 100 | 100 | 87.5 | 100 |
| 16 | 12.5 | 100 | 100 | 87.5 | 100 |
| 16 | 0 | 100 | 100 | 87.5 | 100 |
| 17 | 0 | 100 | 100 | 87.5 | 100 |
| 17 | 0 | 100 | 100 | 87.5 | 100 |
| 18 | 0 | 100 | 100 | 87.5 | 100 |
| 18 | 0 | 87.5 | 100 | 87.5 | 100 |
| 19 | 0 | 87.5 | 100 | 87.5 | 100 |
| 19 | 0 | 75 | 100 | 75 | 100 |
| 20 | 0 | 75 | 100 | 75 | 100 |
| 20 | 0 | 75 | 100 | 50 | 100 |
| 21 | 0 | 75 | 100 | 50 | 100 |
| 21 | 0 | 75 | 100 | 37.5 | 100 |
| 22 | 0 | 75 | 100 | 37.5 | 100 |
| 22 | 0 | 75 | 100 | 37.5 | 100 |
| 23 | 0 | 75 | 100 | 37.5 | 100 |
| 23 | 0 | 75 | 100 | 37.5 | 100 |
| 24 | 0 | 75 | 100 | 37.5 | 100 |
| 24 | 0 | 62.5 | 37.5 | 12.5 | 100 |
| 25 | 0 | 62.5 | 37.5 | 12.5 | 100 |
| 25 | 0 | 25 | 0 | 0 | 87.5 |
| 26 | 0 | 25 | 0 | 0 | 87.5 |
| 26 | 0 | 25 | 0 | 0 | 75 |
| 27 | 0 | 25 | 0 | 0 | 75 |
| 27 | 0 | 12.5 | 0 | 0 | 62.5 |
| 28 | 0 | 12.5 | 0 | 0 | 62.5 |
| 28 | 0 | 12.5 | 0 | 0 | 50 |
| 29 | | 12.5 | | | 50 |
| 29 | | 12.5 | | | 50 |
| 30 | | 12.5 | | | 50 |
| 30 | | 12.5 | | | 50 |
| 31 | | 12.5 | | | 50 |
| 31 | | 12.5 | | | 50 |
| 32 | | 12.5 | | | 50 |
| 32 | | 0 | | | 37.5 |
| 33 | | | | | 37.5 |
| 33 | | | | | 37.5 |
| 35 | | | | | 37.5 |
| 35 | | | | | 37.5 |
| 40 | | | | | 37.5 |
| 40 | | | | | 37.5 |
| 41 | | | | | 37.5 |
| 41 | | | | | 0 |
| 42 | | | | | |
| 42 | | | | | |
| 84 | | | | | |
| Mean Survival | 14 | 24 | 23 | 19.5 | 29 |
| (days) | | | | | |

TABLE 5
Statistical Significance

| | Day 0- | Day 0- | Day 0- | Day 0- | Day 0- | Day 5- | Day 5- | Day 5- | Day 5- | Day 5- |
|---|---|---|---|---|---|---|---|---|---|---|
| | Placebo | 28% | 2.2% | 0.2% | Systemic | Placebo | 28% | 2.2% | 0.2% | Systemic |
| Day 0- | — | 0.0001 | 0.0001 | 0.0014 | 0.0001 | 0.5919 | 0.0001 | 0.0001 | 0.0006 | 0.0001 |
| Placebo | | | | | | | | | | |
| Day 0- | 0.0001 | — | 0.0434 | 0.0069 | 0.7869 | 0.0001 | 0.0075 | 0.0021 | 0.0006 | 0.9992 |
| 28% | | | | | | | | | | |
| Day 0- | 0.0001 | 0.0434 | — | 0.6328 | 0.2237 | 0.0001 | 0.2395 | 0.0008 | 0.0002 | 0.1250 |
| 2.2% | | | | | | | | | | |
| Day 0- | 0.0014 | 0.0069 | 0.6328 | — | 0.4226 | 0.0004 | 0.6756 | 0.2283 | 0.0442 | 0.2137 |
| 0.2% | | | | | | | | | | |
| Day 0- | 0.0001 | 0.7869 | 0.2237 | 0.4226 | — | 0.0001 | 0.0142 | 0.0008 | 0.0002 | 0.5157 |
| Systemic | | | | | | | | | | |
| Day 5- | 0.5919 | 0.0001 | 0.0001 | 0.0004 | 0.0001 | — | 0.0001 | 0.0001 | 0.0004 | 0.0001 |
| Placebo | | | | | | | | | | |
| Day 5- | 0.0001 | 0.0075 | 0.2395 | 0.6756 | 0.0142 | 0.0001 | — | 0.3377 | 0.0275 | 0.222 |
| 28% | | | | | | | | | | |
| Day 5- | 0.0001 | 0.0021 | 0.0008 | 0.2283 | 0.0008 | 0.0001 | 0.3377 | — | 0.0257 | 0.003 |
| 2.2% | | | | | | | | | | |
| Day 5- | 0.0006 | 0.0006 | 0.0002 | 0.0442 | 0.0002 | 0.0004 | 0.0275 | 0.0257 | — | 0.0001 |
| 0.2% | | | | | | | | | | |
| Day 5- | 0.0001 | 0.9992 | 0.1250 | 0.2137 | 0.5157 | 0.0001 | 0.222 | 0.003 | 0.0001 | — |
| Systemic | | | | | | | | | | |

As demonstrated previously, no toxicity was observed in any rats that received locally implanted rapamycin beads. Additionally, none of the rats that received systemic injections of rapamycin showed overt signs of toxicity. The placebo beads implanted on Day 0 and on Day 5 had no effect on survival. All groups that received rapamycin, either by locally delivered beads or by systemic injection, did statistically better than control placebo animals.

There was a dose response seen with animals that received treatment on Day 0—the 28% rapamycin bead animals lived significantly longer than the 2.2% and 0.2% groups. Similarly, in the established tumor model the 28% and 2.2% rapamycin bead groups lived significantly longer than the 0.2% group. There was also a significant benefit seen when treatment was given simultaneously with tumor as opposed to with established tumor. All of the locally treated rapamycin groups that received treatment simultaneous with tumor lived significantly longer than those treated five days after tumor implantation.

In both simultaneous treatment (Day 0) and established tumor treatment (Day 5) there was no significant difference between animals that received the 28% rapamycin beads and those receiving systemic rapamycin. This, however, is interesting in that the 28% beads delivered a total dose of 3 mg rapamycin, whereas the systemically treated animals received a total of 15 mg of rapamycin over the course of administration. The 2 mg/kg daily IP dose used here is well within the doses known to cause clinically significant immunosuppression in rodents (Saunders, R N, Metcalfe, M S, Nicholson, M L, 2001, Kidney Intl., 59:3-16). Therefore, it is anticipated that local intracranial delivery of rapamycin will provide the anti-tumor effect of systemically delivered drug without the potentially life threatening immunosuppressive side effects.

### EXAMPLE 7

### Human Surgery

A patient with a glioblastoma is prepared for cranial surgery in a conventional manner. Surgery is a preferred standard treatment for brain tumors. The surgeon performs a conventional craniotomy and resection to remove the tumor from the patient's brain. A section of bone (bone flap) is removed from the skull so that the underlying tissue can be accessed for the surgical procedure. The bone flap is replaced at the end of the procedure. To the greatest extent possible, the surgeon removes the entire tumor, while minimizing any damage to adjacent tissue. If the tumor cannot be completely removed without damaging vital areas of the brain, the surgeon will remove the tumor to the extent possible. The surgeon then administers to the resection site a therapeutically effective amount of the compressible hollow foam bead(s) of the present invention loaded with a chemotherapeutic agent to sufficiently fill the cavity at the resection site. The compressible hollow foam bead(s) are administered to the glioblastoma resection site by first compressing the bead(s), then placing the bead(s) in the cavity of the site, and allowing the bead(s) to resume to their original spherical shape or a shape substantially conforming to the surface of the resection site or possibly to remain in a compressed state, or a combination thereof. The beads adjacent to the tissue surrounding the cavity of the resection site will substantially conform to the contours of the cavity. Although it is preferred to fill in the entire cavity, the surgeon may in the surgeon's discretion fill in a part of the cavity. The surgical site is then closed in a conventional manner. The patient is optionally treated with conventional radiation therapy and/or chemotherapy, in particular at the resection site and surrounding tissue. Post-surgery, the patient may optionally receive steroids to help reduce swelling, antiepileptic medications to control seizures, and antibiotics to fight infection, and other conventionally administered therapies and treatments.

Although preferred embodiments of the present invention have been described and illustrated, it will be apparent to those skilled in the art that various modifications may be made without departing from the principles of the invention.

We claim:

1. A compressible hollow foam bead comprising a biocompatible, bioabsorbable elastomeric copolymer and at least one chemotherapeutic agent.

2. The bead of claim 1, wherein the chemotherapeutic agent comprises a therapeutically effective amount.

3. A method of making a compressible hollow foam beads, comprising the steps of:
preparing a solution of a biocompatible, bioabsorbable elastomeric copolymer in a solvent,
adding a sufficiently effective amount of a chemotherapeutic agent to said solution;
adding drops of the solution to a liquid nitrogen bath to provide hollow frozen drops, and
lyophilizing said frozen drops to provide a compressible hollow foam beads containing the chemotherapeutic agent.

4. The method of claim 3, wherein the beads comprise a therapeutically effective amount of the chemotherapeutic agent.

5. A method for treating a tumor, comprising:
resecting a tumor at a site in a body, thereby forming a cavity at the site;
providing at least one compressible hollow foam bead comprising a biocompatible, bioabsorbable elastomeric copolymer and at least one chemotherapeutic agent; and,
administering to the cavity at the resection site a therapeutically effective amount of one or more of the compressible hollow foam beads to substantially fill all or part of the resection site.

6. The method of claim 5, wherein the tumor is a glioblastoma.
